(19) United States
(12) Patent Application Publication
(10) Pub. No.: US 2014/0364711 A1
(43) Pub. Date: Dec. 11, 2014

(54) ALL-IN-ONE ANALYTE SENSOR IN A DETACHABLE EXTERNAL MOBILE DEVICE CASE

(71) Applicant: AkibaH Health Corporation, Alexandria, VA (US)

(72) Inventors: Moussa Omar Ismail, Alexandria, VA (US); Haroon Ismail, Alexandria, VA (US); Aaqib Mohammed Habib, Pittsford, NY (US); Fahad Masood, Henrietta, NY (US)

(21) Appl. No.: 14/203,573

(22) Filed: Mar. 11, 2014

Related U.S. Application Data
(60) Provisional application No. 61/805,861, filed on Mar. 27, 2013.

(51) Int. Cl. A61B 5/145; A61B 5/00; A61B 5/1486; A61B 5/151

(52) U.S. Cl. CPC A61B 5/14532; A61B 5/151; A61B 5/0015; A61B 5/742; A61B 5/1486
USPC 600/365

(57) ABSTRACT

The embodiments herein discuss an analyte sensing processor housed in the external, detachable case of a mobile device. The analyte sensing processor converts the reading into a signal that can be further processed by a health management system and displayed on the graphical user interface of a user's device.

### CROSS-REFERENCE TO RELATED APPLICATIONS

This application claims the benefit of U.S. Provisional Application No. 61/805,861, filed Mar. 27, 2013.

### BACKGROUND

The present invention is in the technical field of analyte sensors and medical devices for portable analyte testing.

The incidence of medical conditions such as diabetes mellitus, hypercholesterolemia, and hypertension is increasing rapidly in developed countries due to increasing obesity, inactive lifestyles and an aging population. As the number of patients suffering from diabetes and similar medical conditions increases, a corresponding increase in diabetes and health and wellness monitoring care will be needed.

The goal of any type of diabetes care is to keep blood glucose levels as normal as possible. Complications of diabetes may be more prevalent if blood glucose is not controlled. Some examples of complications are high blood pressure, stroke, eye disease/blindness, kidney disease, heart disease, foot disease and amputations, complications of pregnancy, skin and dental disease. In order to keep blood glucose levels normal, diabetics require regular feedback regarding their current blood glucose levels. This feedback will provide guidance on how to improve future readings, thereby providing a positive educational experience that will influence their long term health.

Most diabetics use glucose meters to check their blood glucose. To test glucose levels with a typical meter, blood is placed on a disposable test strip and placed in the meter. The test strips are coated with suitable chemicals, such as glucose oxidase, dehydrogenase, or hexokinase that combine with glucose in the blood. The meter measures how much glucose is present based on the reactions with these chemicals.

Blood glucose meters often further include a memory for storing measured blood glucose values, exercises and meals, along with other related data such as the corresponding dates, time of day, and duration of each, and the units that were used as these values and events were measured. Blood glucose meters are also generally provided with a display screen and user input buttons or controls with which a user can specify which of the stored values to display or functions to access.

A blood glucose meter can be configured to receive and read an inserted test strip on which a drop of a patient's blood has been deposited. Many current devices include a plethora of separate components in order to facilitate self-monitoring. Such systems are disclosed, for example, in U.S. Patent Publication 20130245660 A1, to Tara Chand Singhal, entitled “Apparatus and methods for a lancet device for reuse of lancets for home-users” and European Patent Application 2484282 A2, entitled “Blood glucose meter capable of wireless communication”; the entire content of both incorporated herein by reference. There are numerous blood glucose meters in the marketplace, but the instruments consume physical space and are not pocketable. The instruments usually have to be carried in a large handbag, or an individual's briefcase, or left at home such as in the bathroom or the bedroom on a counter or table.

The measurement of blood glucose levels is preceded by a preparation process that involves the patient lancing themselves with a lancet and impregnating a blood glucose test strip with a blood sample. The number of devices necessary to obtain a blood glucose level reading is many; thus, requiring users to carry many devices in separate, often bulky and obtrusive, containers. Likewise, patients who check their cholesterol levels frequently find it inconvenient to carry around a bulky apparatus.

Therefore, a better mode is required to carry all the components and accessories of a blood glucose meter, or any analyte sensing device, in an ergonomic and compact manner.

Additionally, many users utilize digital diabetes management systems to track their blood glucose levels and monitor their condition graphically (e.g., U.S. Pat. No. 7,862,506). However, many users find this process, which often involves data entry, to be tedious and unintuitive. Thus, the method of conveying and displaying information in the digital diabetes management system should be more intuitive and actionable for the growing demographic of people using them.

In order to solve these problems, there is a need for the development of an all-in-one, compact analyte sensor which can take advantage of the application and display of a smart phone and which can be externally combined with the smart phone and housed in a smart phone case. A smart phone is an intelligent terminal in which computer support functions such as Internet communication and information searching have been added to a mobile phone, and is a portable communication device on which a user can install desired applications. Accordingly, when an analyte sensor such as a blood glucose measurement device having the minimum number of elements required to measure and calculate the blood glucose level is combined with and then used in conjunction with a smart phone, the problems of the above-described conventional technology can be considerably surmounted.

### SUMMARY

According to one embodiment of the invention, an analyte sensor comprising an external mobile device case, incorporating the minimum number of elements required to measure and calculate blood glucose concentration and/or other component concentrations (e.g., cholesterol, inter alia), is attached to a mobile device. Suitable mobile devices include, but are not limited to, devices such as the Apple iPhone™, the Apple iPod™, and Android™ mobile devices. The external mobile device case can operate in conjunction with the attached mobile device as a fully-functional all-in-one analyte sensor. Additionally, according to another embodiment of the invention, the external mobile device case can operate, without being attached to a mobile device, as a standalone, fully-functional all-in-one analyte sensor.

In one embodiment of the invention, an external mobile device case houses the following: a lancet ejector cartridge which contains a plurality of lancets; a test strip storage cartridge which contains a plurality of electrochemical test strips; and an analyte sensing processor. An analyte sensing processor is a device that measures various component concentrations, e.g., blood glucose, cholesterol, etc. (not an exhaustive list).

The analyte sensing processor measures the electrochemical property of a blood sample and forwards the reading to a mobile device. The reading can then be stored locally on the mobile device or sent to a remote storage or cloud database via a network, such as the Internet. The aggregate data can then be used instantaneously, or at a later time, for complex analyses or presented to the user through the graphical user interface of the mobile device and/or through other interface means via other electronic devices, such as, but limited to, smart phones, personal computers, personal electronic computing devices, smart watches, smart glasses, smart accessories, inter alia.

A health management system can be comprised of hardware, software, or a combination of both. Said system can act as an engine and repository for raw biomarker data which can later be processed, analyzed, and interpreted by the health management system to provide the user with personalized suggestions. In one exemplary embodiment, the health management system can include a glucose monitoring application downloaded and embedded in a smart phone, or other mobile device, which stores results of the glucose measurements locally and/or in a personalized cloud database to be accessed by the user and shared with physicians, emergency personnel, insurance providers, friends, or family members if needed through automated phone calls, SMS/text, or emails. The glucose monitoring application can process the dietary and fitness actions of the user and give personalized suggestions to maintain a healthy lifestyle and/or attain desired health and fitness goals.

### BRIEF DESCRIPTION OF THE DRAWINGS

FIGS. 1 and 2 are perspective views schematically showing the appearance of an analyte sensor according to example embodiments of the present invention.

FIG. 3 illustrates a side view showing the appearance of an analyte sensor according to example embodiments of the present invention.

FIG. 4 illustrates a bottom view showing the appearance of an analyte sensor according to example embodiments of the present invention.

FIG. 5 is a flowchart schematically outlining an analyte measurement method using a smart phone which can be combined with the analyte sensor according to example embodiments of the present invention.

FIGS. 6-13 illustrate screenshots of health management system applications according to example embodiments of the present invention.

FIG. 14 illustrates one aspect of the health management system according to example embodiments of the present invention, including a network, computers, servers, and a local and cloud database.

### DETAILED DESCRIPTION

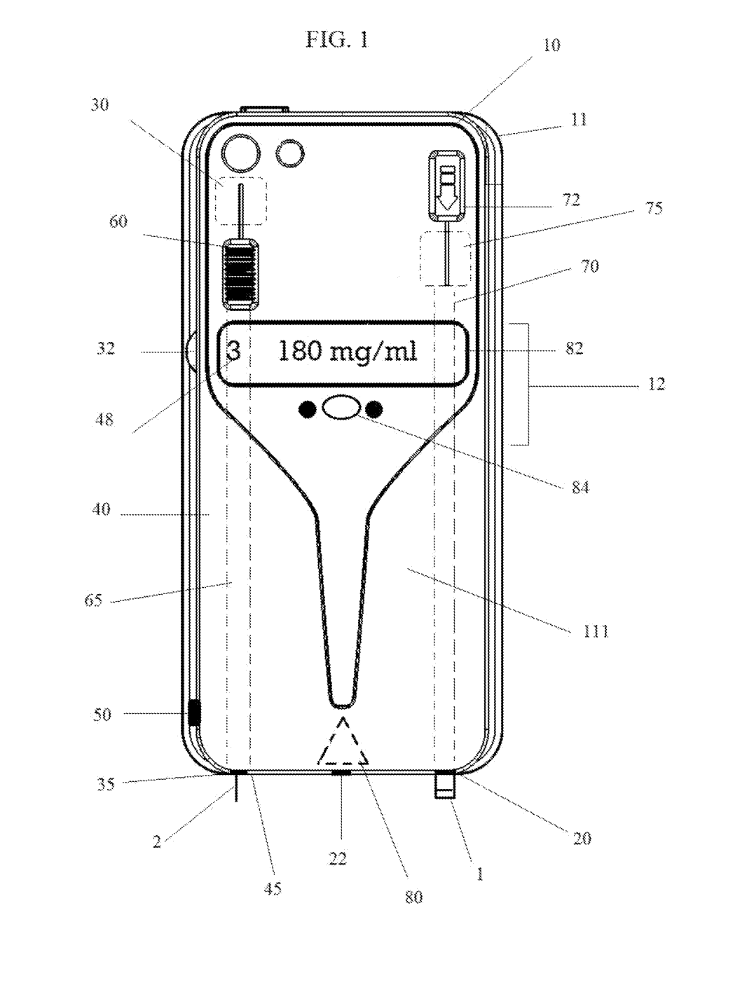

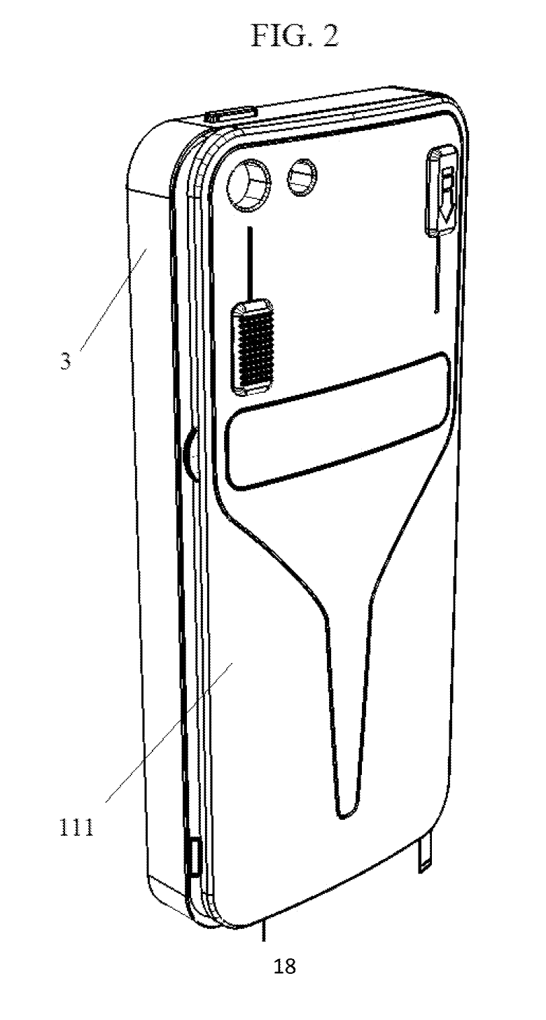

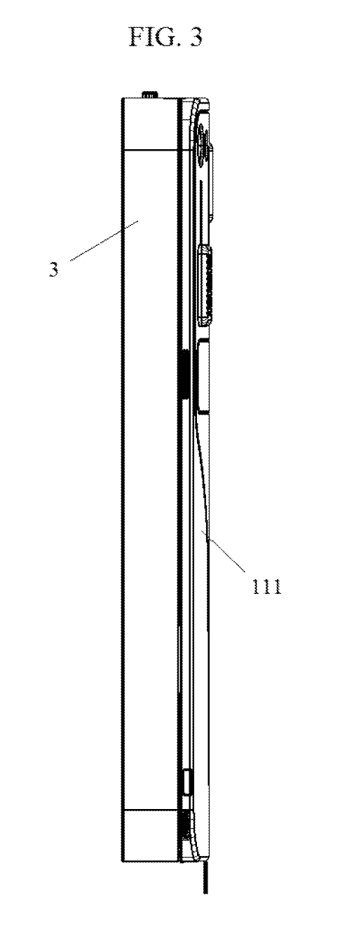

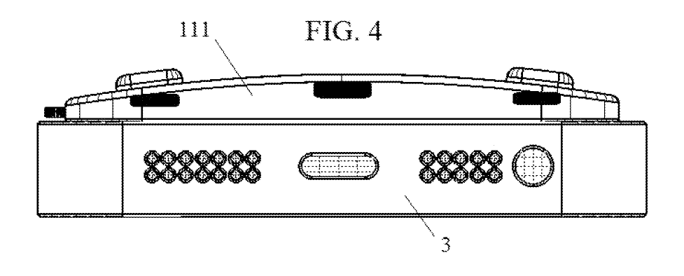

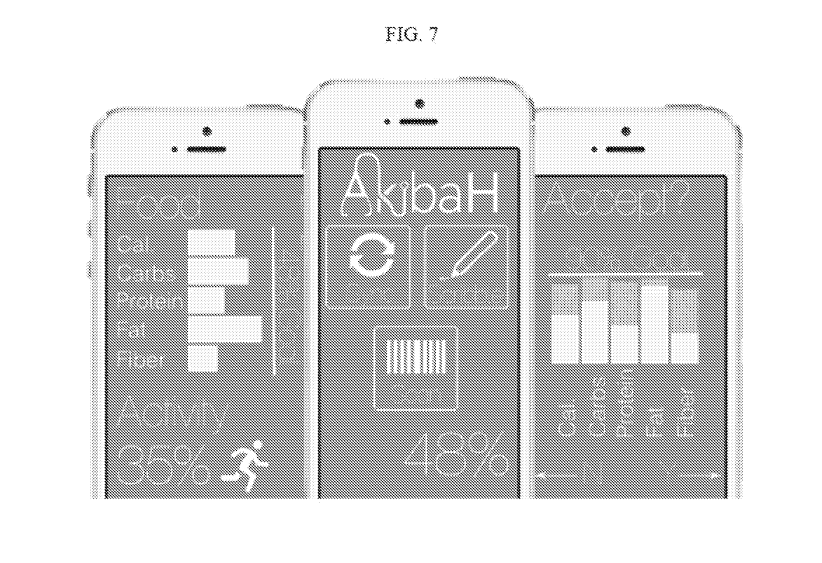

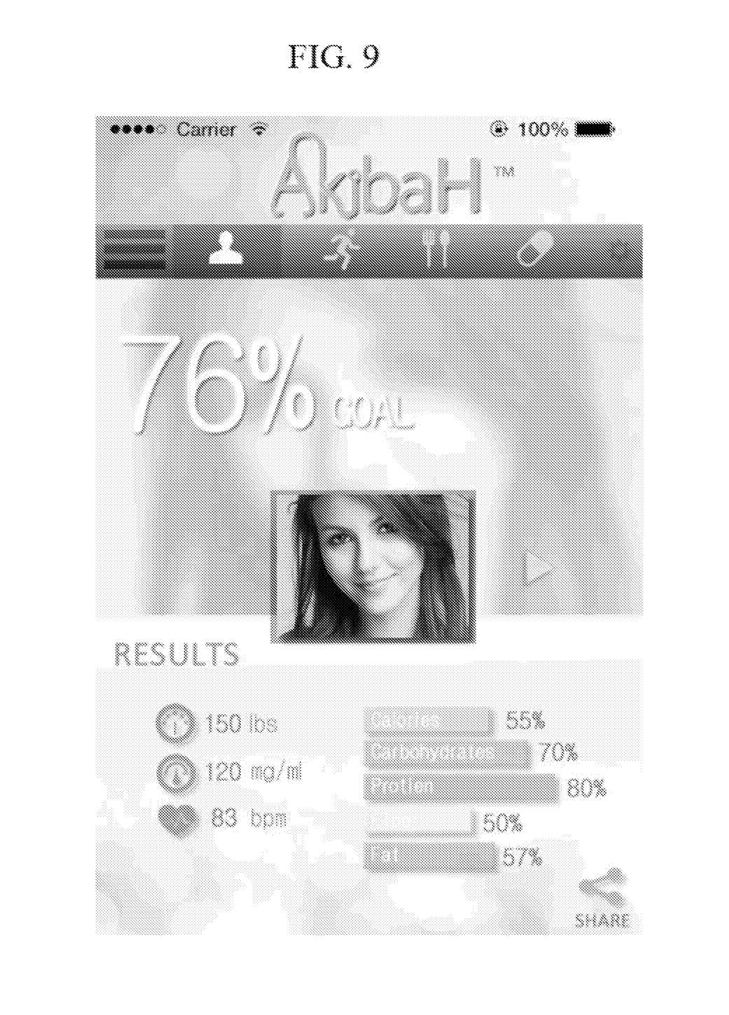

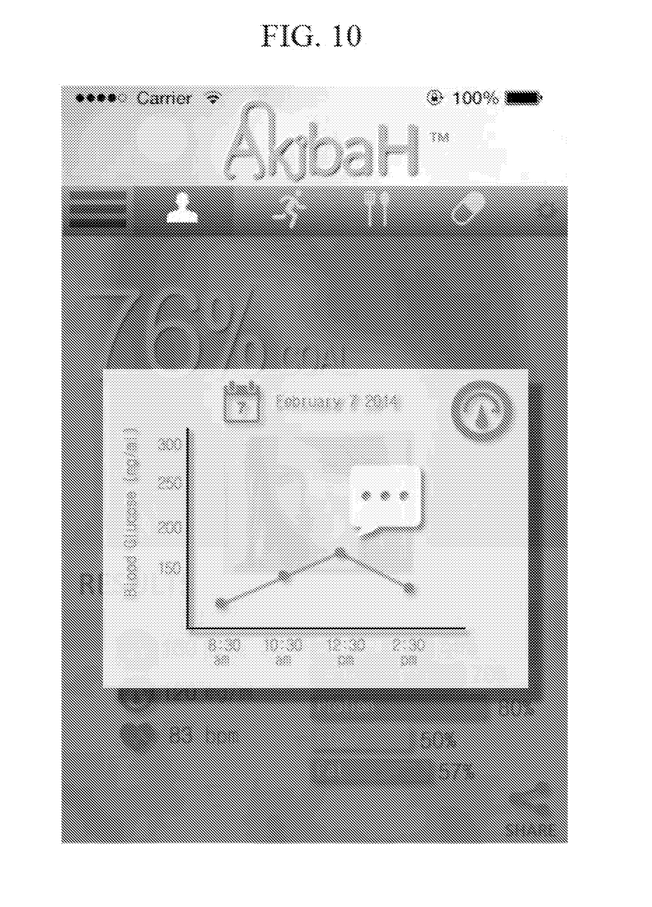

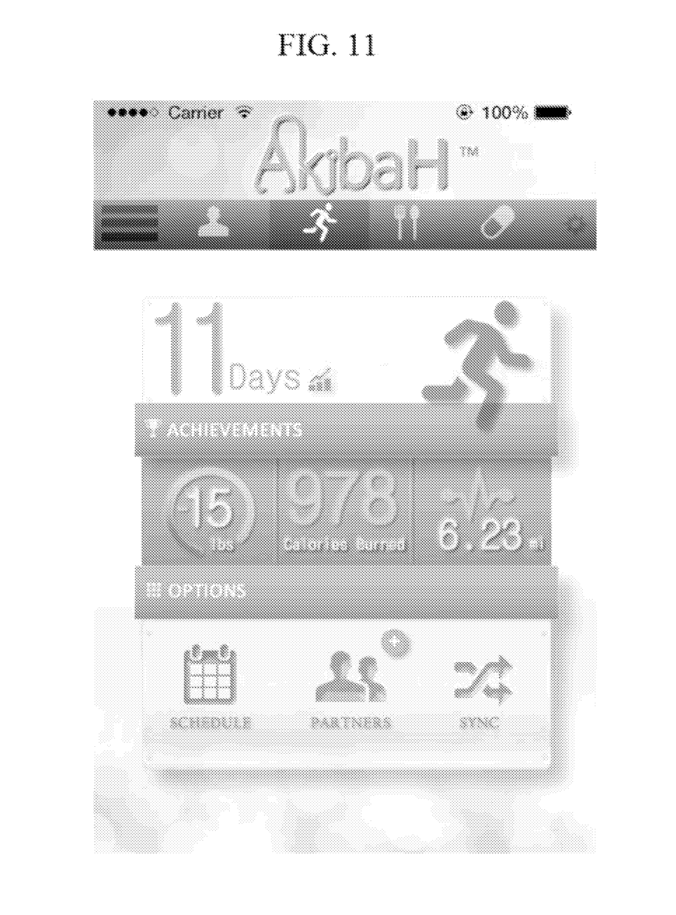

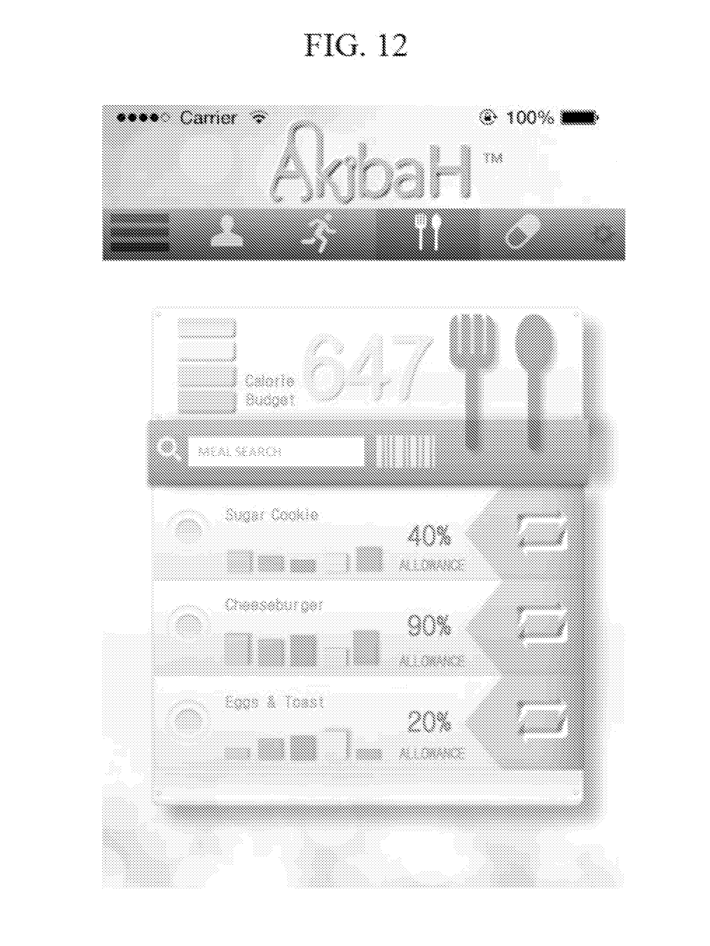

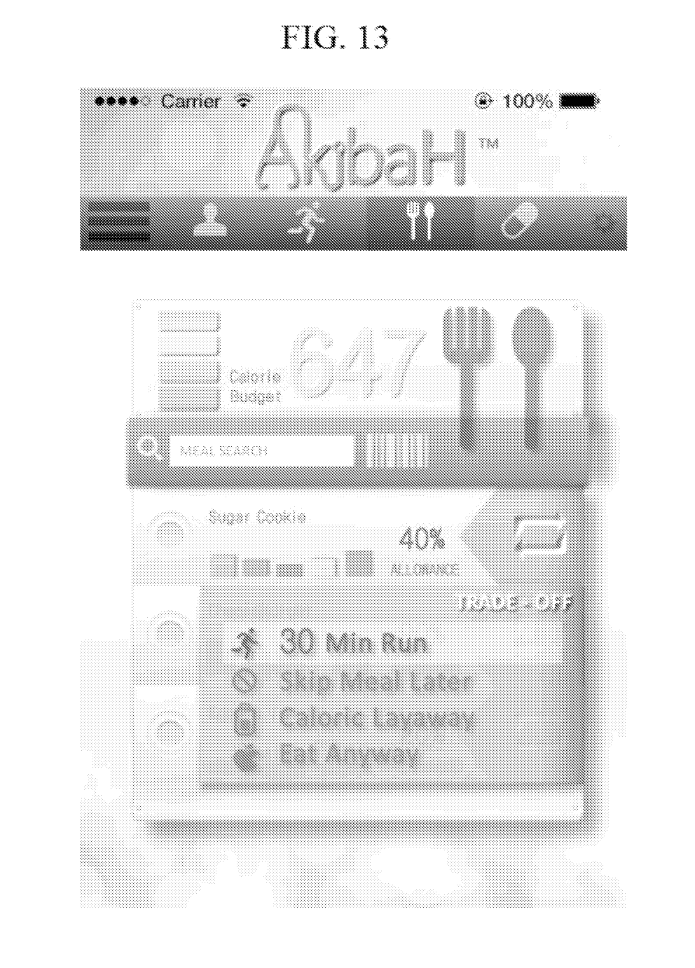

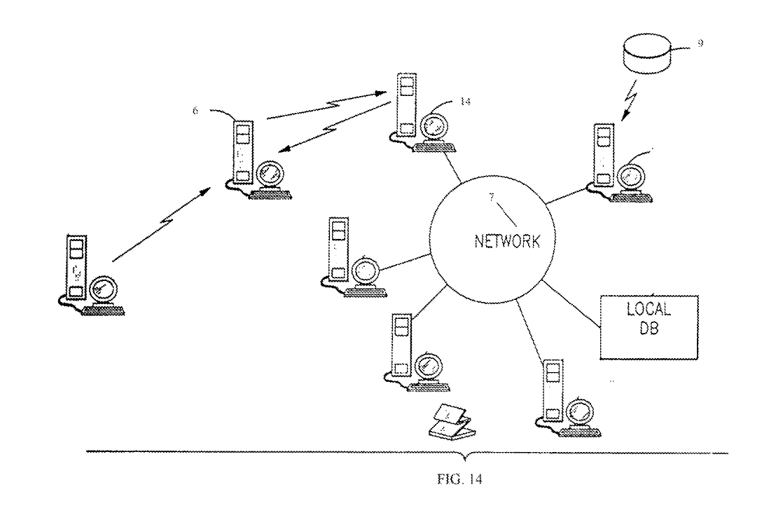

The following will now make reference to an embodiment of the invention. FIGS. 1-14 will be referenced, in detail, as examples illustrating said embodiment. Similar elements within drawings will maintain uniform reference numerals.

The exemplary embodiments of the present invention described below relate to a blood glucose meter housed in a mobile device case with an integral lancet ejector cartridge containing multiple lancets, and a location within the mobile device case for a test strip storage cartridge containing multiple, ejectable electrochemical test strips, an analyte sensing processor, and a display that may be configured to display the test results, as well as other information. The display may be a LCD display or any other electronic display capable of displaying blood-sugar level test results, such as a LED display. The analyte sensing processor 80 can be a Central Processing Unit (CPU), Microprocessor (MCU), or Microcontroller for calculating and transmitting results of the measurement of the blood glucose concentrations as well as a plurality of other component concentrations (e.g., cholesterol, inter alia). The disclosed embodiments of the present invention combine the functionality of each above feature into a streamlined enclosure that optimizes the use of the product for the purpose of monitoring one's blood glucose and/or other components.

As noted above, many existing devices require the use of a separate blood glucose meter, lancet device and test strip storage vial. These existing devices can, therefore, require an extensive amount of handling and manipulation of separate devices to facilitate the measurement of one's blood glucose, which is an undesirable outcome. The exemplary embodiments of the present invention combine these separate device features into a single device by combining a blood glucose meter, analyte sensing processor, lancet device and test strip storage cartridge, thereby requiring much less handling and manipulation to accomplish a desirable outcome.

FIG. 1 is a perspective view illustrating the top surface of an analyte sensor 111 in accordance with an embodiment of the present invention. The main features of the disclosed embodiments of the present invention include a device body 10 configured for convenient use, test strip exit port 20 and lancet port 35 that are disposed at the same end of the device body 10, thereby allowing a drop of blood extracted by the lancet 2 to be immediately deposited on the test strip 1 ejected from the test strip exit port 20, a detachable cover 40 which allows lancet and test trip replacement and also provides a generous lead-in area 45 for lancing a surface of skin, a test strip entrance port 22 to facilitate convenient test strip loading for processing, a trigger button 50 on the side of the device body 10 which allows comfortable positioning during lancing, a lancet arming slide 60 on the side of the enclosure of the device body 10 which arms the lancing mechanism of the lancet ejector cartridge 30 when moved toward the top of the device, and a test trip storage cartridge 75 containing multiple, ejectable test strips. The device body 10 further includes an analyte sensing processor 80 for processing the test strip. The device body 10 can further include a display window 82 and a plurality of meter operation buttons or controls 84.

By combining these multiple components into a single device body 10, the device requires fewer steps for testing, and makes device use easier, even in confined or less than ideal locations to test one's blood glucose levels. The analyte sensor 111 can be a blood glucose measurement system, a cholesterol measurement system, or measurement system for a plurality of various single component concentrations or multiple component concentrations. In other implementations, the analyte sensor may be any device that may be configured to determine the level of one or more analytes (e.g., ketones, cholesterol, lactate, and the like). Nothing within the following description however will limit the diagnostic device to a blood glucose meter unless such context is so limiting, as other diagnostic devices are contemplated and do not depart from the scope of this disclosure.

The embodiment of the present invention shown in FIG. 1 takes advantage of the small size of the primary sub-components, including blood glucose sensing circuitry/technology of the blood glucose meter 12, lancet mechanism of the lancet ejector cartridge 30, and test strip storage cartridge 75, and encapsulates each in an attractive and user-friendly package. The combination of these sub-systems as shown in the embodiment of FIG. 1 results in a reduction in the number of steps required to test one's blood glucose as described in greater detail below.

The analyte sensor 111 of FIG. 1 includes a device body 10 which is configured for convenient portable use. The device body 10 is further housed by a rounded device bumper 11 hugging the device body 10 as well as the attached mobile device 3. The device body 10 can be any suitable length, but preferably comprises a length of about 127 mm. The top and bottom surfaces of the device body 10 preferably have a width of about 56 mm, and a depth of about 5 mm. The device body 10 and device bumper 11 can be constructed of any suitable material, but is preferably constructed of a flexible engineering plastic material. In one embodiment, the analyte sensor 111 also includes a battery. Preferably, the battery is electrically connected to the power signal at a switch. The switch controls whether the battery provides power to a microcontroller system and peripheral devices in the analyte sensor. In another embodiment, to reduce the number of physical ports required in the analyte sensor, a multiplexing module, such as an MC34825 from Freescale Semiconductor, Inc., is connected to a USB port. The multiplexing module can allow the USB port to be used for USB purposes, for a non-USB serial interface, and for an audio interface. In addition, a power supply can be connected to the USB port. The power supply \ can include a battery, such as a lithium ion rechargeable battery, which provides power to components of the handheld diabetes management device. The power supply can be recharged via the USB port.

At a distal end of the device of FIG. 1, a test strip exit port 20, test strip entrance port 22, and lancet port 35 are disposed at the same end of the device body 10. In doing so, a test strip 1 can be provided very close to the lancet port 35. The test strip exit port 20 and test strip entrance port 22 can be provided to be within 40 mm of the distal end of the lancet port 35. A detachable cover 40 is provided and allows convenient lancet replacement when desired. The detachable cover 40 can be constructed of any suitable material, but is preferably constructed of the same material as the device body 10. The detachable cover 40 can be secured to the device body 10 using any number of attachment mechanisms, such as a snap-fit mechanism.

A trigger button 50 is disposed on one end of the device body 10, allowing comfortable positioning during lancing. The trigger button 50 is mechanically engaged with the lancet mechanism of the lancet ejector cartridge 30 through the device body 10 to activate the lancet as known to those skilled in the art when a force is exerted on the trigger button 50, such as when pressed by a user. An arming slide 60 is disposed on the left side of the device body 10 to minimize the overall envelope of the device. The arming slide 60 is also mechanically engaged with the lancet mechanism of the lancet ejector cartridge 30 through the device body 10 to arm the lancet as known to those skilled in the art through a sliding motion of the arming slide 60, such as when slid by a user. Accordingly, the arming slide 60 can be disposed within a recess 65 extending over the side of the device body 10. The arming slide 60 can be guided in the recess 65 using any number of mechanisms, such as rails (not shown) disposed along each side of the recess 65 and engaged by the arming slide 60. By further providing the arming slide 60 with a number of raised members 62, a user can firmly grasp the arming slide 60 and arm the lancet by pressing the proximal end of the device body 10 against a surface to move the arming slide 60 within the recess 65.

The analyte sensor 111 further includes a test strip storage cartridge 75 at the right of center end of the device body 10 which houses a plurality of test strips, and which can be accessed by removing the detachable cover 40. A recess enclosure 70 having sufficient diameter and which extends to a sufficient depth to receive a test strip 1 ejected from a test strip storage cartridge 75. In an exemplary embodiment of the present invention, the recess enclosure 70 can have an opening of 18 mm at the bottom of the device and a depth of 100 mm running up the right vertical end of the device 10. The recess enclosure 70 can further comprise a spring-loaded retention feature for the test strip storage cartridge 75, such as a mechanical locking and spring-loading ejection mechanism or rails (not shown), for engaging and retaining the test strip storage cartridge 75 therein.

Similar to the lancet arming slide 60, test strip ejector slide 82 is disposed on the right side of the device body 10 to minimize the overall envelope of the device. The test strip ejector slide 82 is also mechanically engaged with the ejection mechanism of the test strip storage cartridge 75 through the device body 10 to eject the test strip through a sliding motion of test strip ejector slide 82, such as when slid by a user. Accordingly, the test strip storage cartridge 75 can be disposed within the recess space of recess enclosure 70 extending over the right side of the device body 10. The test strip 1 can be guided in the recess enclosure 70 using any number of mechanisms, such as a spring-loading ejection mechanism or rails (not shown) disposed along each side of the of recess enclosure 70 and engaged by the test strip ejector slide 82. By further providing the test strip ejector slide 82 with a number of raised members, a user can firmly grasp the test strip ejector slide 82 and dispense test strips by sliding the test strip ejector slide 82 to eject test strips within the recess enclosure 70.

The device body 10 further includes a lancet ejector cartridge 30 for lancing a skin surface and providing a blood sample to a test strip 1 held in the test strip exit port 20. The tip of the lancet ejector cartridge 30 comprises a substantially cylindrical depth control mechanism 32 against which the user engages a skin surface. Accordingly, the lancet ejector cartridge 30 can be adjustable. In the embodiments of the present invention, the depth setting is selected by rotating the cylindrical depth control mechanism 32 to the desired setting number positioned adjacent to the depth selection indicator 48. Further, the lancet ejector cartridge 30 can be armed and activated as described above, and can include lancets that can be easily accessed via the detachable cover 40.

The device body 10 further includes an analyte sensing processor 80 for processing the test strip 1 received via the test strip entrance port 22. The device body 10 can further include a display window 82, such as an LCD display or like device, which can display any number of test results. A plurality of analyte sensor operation buttons or controls 84 can be provided to allow a user to control the analyte sensing processor 80 and meter display window 82. The test results are then transmitted to the mobile device 3 via direct electronic connection (via, for example, the 30 pin iPod™ proprietary connector or USB connection) or wireless connection (via, for example, Wi-Fi, Bluetooth™, or Bluetooth™ Low Energy (BLE)). The mobile device 3 may then communicate the results to a server 14 via the cellular telephone network or via a http protocol using a wireless local area network or by some other communication means to another network. The test results (e.g., 1-day averages, 7-day averages, 30 day averages) may be compiled and/or calculated on the blood glucose meter 12, the mobile device 3 or at the server 14 level. Feedback to the user can be given and displayed by the blood glucose meter 12 via the display window 82, the mobile device 3, or both. The feedback can be in the form of text, images, audio, and/or video among other forms of visual, audible, and/or tactile feedback. Of course, such a system can also be regarded as comprising an sensor as described above with or without a wireless communication module connected to the analyte sensor as well as a number or set of wireless communication modules which are adapted for different wireless communication capabilities. This set of wireless communication modules may e.g. include wireless communication modules adapted to communicate according to the ISM, Bluetooth, ZigBee or WLAN standard or even according to two or more of these standards. In view of BLE, as compared to “Classic” Bluetooth, BLE provides considerably reduced power consumption and cost while maintaining a similar communication range.

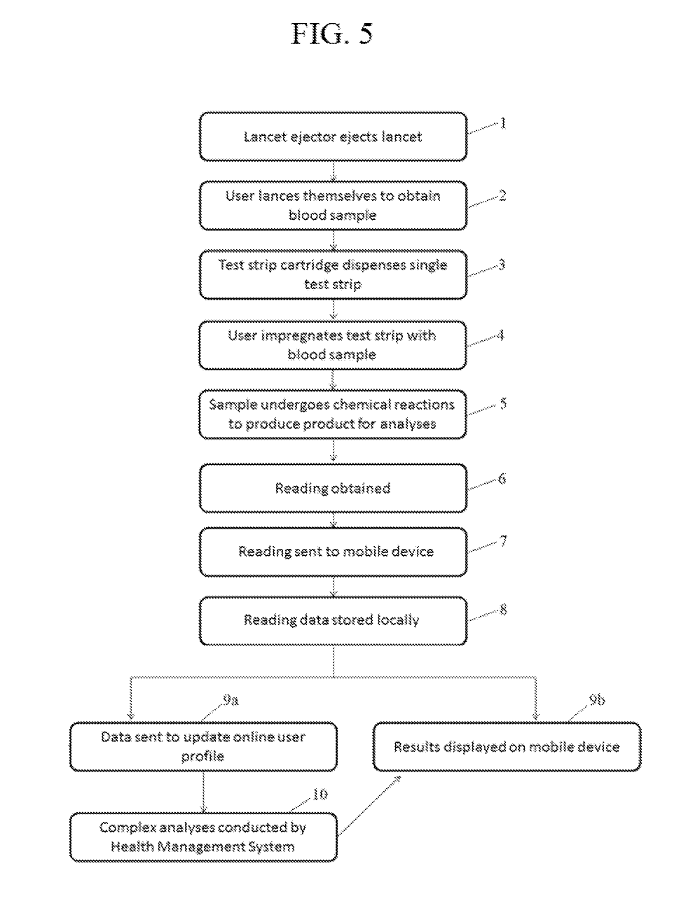

The use of the disclosed embodiments of the present invention, described in greater detail below, significantly benefits from the combined features described above and shown in FIGS. 1-4. As noted above, the embodiments of the present invention include an analyte sensor 111 with an analyte sensing processor 80, an integral lancet ejector cartridge 30, and a test strip storage cartridge 75 to store the test strips that holds a number of test strips (i.e., up to 25 or more). The embodiments further include the test strip exit port 20 and test strip entrance port 22 for the glucose test strip, and the tip of the lancet 2 of the integral lancet ejector cartridge 30, at the same end of the device body 10. In doing so, the embodiments of the present invention allow a user to arm the lancet ejector cartridge 30, lance a finger placed in the lead-in area 45, slide test strip ejector slide 72 which triggers test strip storage cartridge 75 to eject test strip 1, collect the blood on the edge of the test strip 1, and insert the test strip 1 into the test strip entrance port 22, with minimal wasted movement and time (i.e., a virtually continuous short motion). Such steps are outlined in FIG. 5.

Many existing devices provide the lancet device at the opposite end from the test strip, requiring the user to perform an awkward maneuver to rotate the unit after lancing. Still other existing devices require either the use of a separate lancet device in the case of meter-only units, or a rotation of the meter in the case of meters with lancet device and test strip port at opposite ends. The embodiments of the present invention solve these problems by placing the test strip exit port 20, the lancet of the lancet port 35, and the test strip exit port 20 in close proximity at the distal end of the device body 10, thereby minimizing wasted motion.

In one embodiment, once the user is ready to check their blood glucose level, they simply eject one of the lancets stored in the lancet ejector cartridge 30, by arming and triggering the lancet arming slide 60, and prick themselves to obtain a blood sample. The user then impregnates an electrochemical test strip 1, ejected from the test strip storage cartridge 75, with a drop of blood. The blood sample then undergoes a series of chemical reactions with an enzyme (such as glucose oxidase) and a mediator molecule (such as ferricyanide) to produce a product.

The analyte sensing processor 80 then runs an electric current through the product from the chemical reactions using the blood sample. The analyte sensing processor 80 measures the change in electrical current to determine the blood glucose concentration and/or other component concentrations (e.g., cholesterol, inter alia), sends a signal with the data from the measurement to the mobile device 3 which then interprets it, the reading is subsequently displayed on the graphical user interface 4 of the mobile device 3 and the display window 82 of the device body 10 to the user and also viewable and accessible on a user's online personal profile 5 via a website portal and network 7.

The user can later access the information (e.g., the blood glucose readings, nutritional and fitness logs, graphs, and metrics, inter alia) from the local device or remote storage via a health management system 9 which can be accessed through the user's mobile device 3 or personal computing device 6 via a network 7. The health management system 9 can be comprised of hardware, software, or a combination of both. A security system can require the user to enter a password, perform a biometric authentication, or other secure access measure.

The health management system can act as an engine and repository for raw biomarker data which can later be processed, analyzed, and interpreted by such to provide the user with personalized suggestions to maintain a healthy lifestyle and/or attain desired health and fitness goals. In one exemplary embodiment, the health management system can include a glucose monitoring application downloaded and embedded within a smart phone, or other mobile device, which stores results of the glucose measurements locally and/or in a personalized cloud database 9 to be accessed by the user and shared with physicians, emergency personnel, insurance providers, friends, or family members if needed through automated phone calls, SMS/text, or emails. The glucose monitoring application can process the dietary and fitness actions of the user by syncing other outside fitness devices and applications, as well as scanning and/or manually entering diet and nutrition logs. Thus, the health management system allows the user to holistically view trends in their blood glucose levels, cholesterol levels, diet, fitness actions, inter alia, allowing them to make better health choices.

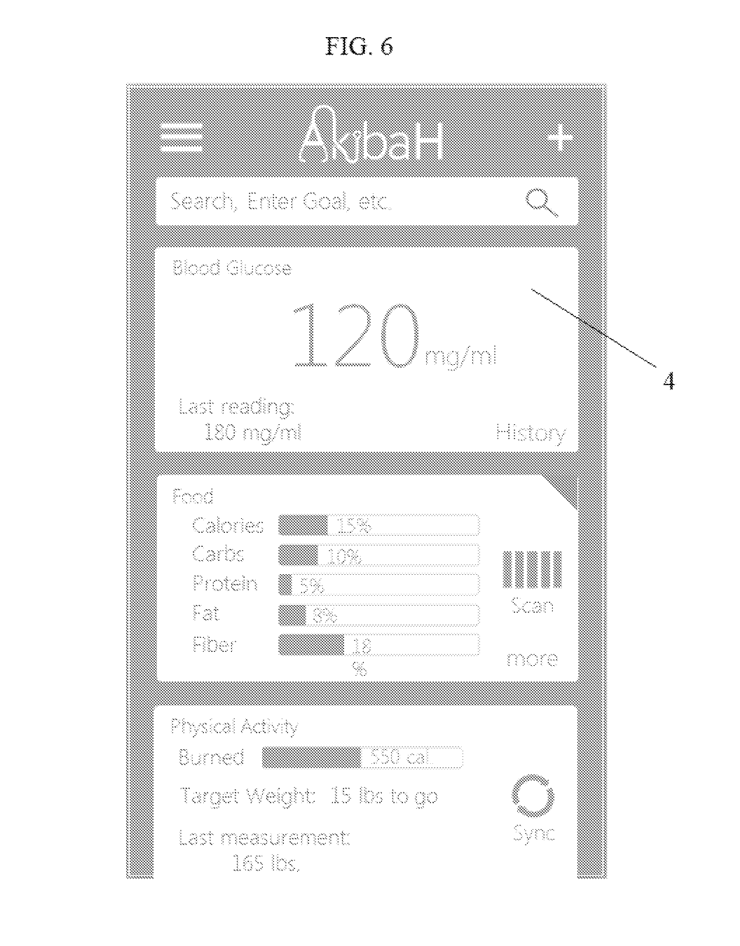

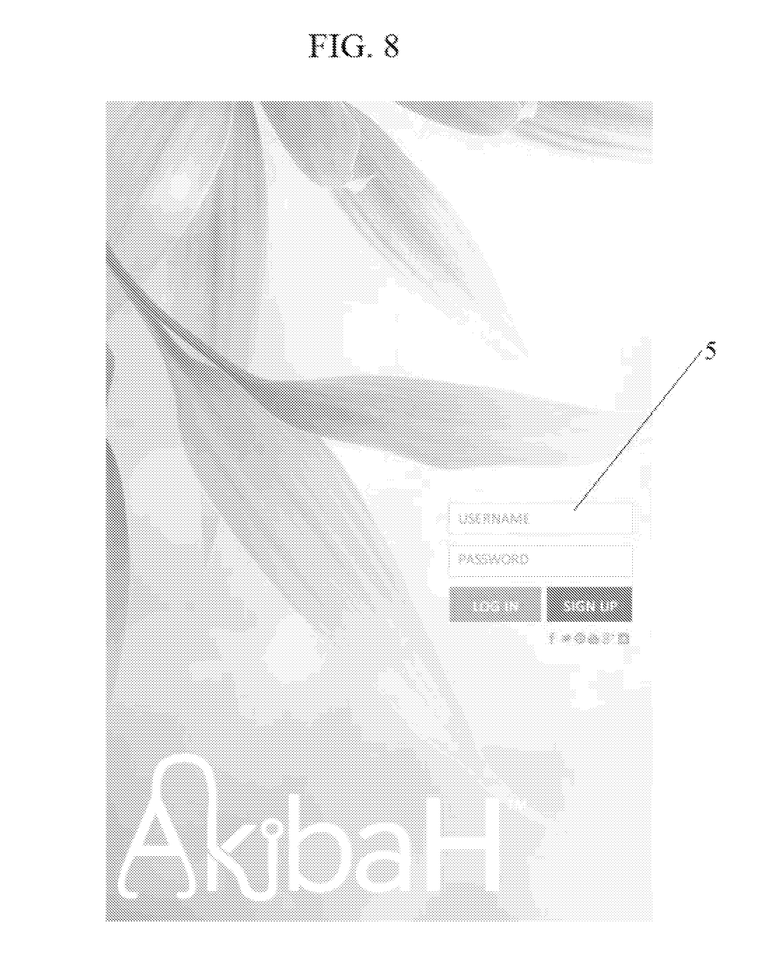

FIGS. 6-13 illustrate screenshots of health management system applications according to example embodiments of the present invention. More specifically, FIGS. 6-13 exemplify several views including displayed metrics and graphs indicating nutrition and fitness logs. Said applications can be used not only by diabetics, but anyone who has an interest in measuring and attaining improvements in health and wellness, including, but not limited to: athletic directors working with athletes off season, quantified-self users looking for actionable data, physicians prescribing “lifestyle medicine” via offering actionable and traceable solutions to get patients from point “A” to point “B.” FIGS. 8-13 illustrate an example process that a user follows when engaging their online personal profile 5, according to one embodiment: 1) User log-ins; 2) User views summarized data on personalized profile (capable of swiping to view friends shared info as well); 3) User clicks on the glucometer icon to display detailed trend info; 4) User views personalized activity data; 5) User views personalized nutrition/diet data; and 6) User expands dietary options to show trade-off suggestions, e.g., if they eat a cookie now, they can later trade-off a 30 minute run exercise for the current consumption of the cookie.

In the use of the embodiments of the present invention as outlined in FIG. 5, a tester can lance any number of positions on a skin surface, such as a bottom surface of a finger or a side surface of a finger. FIG. 5 exemplifies a lancing technique on a surface of a finger. Many testers prefer to simply move the lanced skin surface from the device tip to apply the blood drop onto the adjacent test strip held in the test strip exit port 20.

As outlined in FIG. 5, a test strip 1 is positioned at the distal end of the device body 10 and adjacent to the lancet port 35 as a user engages the lancet with a skin surface. The lancet ejector cartridge 30 includes a substantially cylindrical depth control mechanism 32 in which the user adjusts the lancet depth to engage the skin surface. Once lanced, the lancet 2 is withdrawn slightly from the skin surface to allow the formation of a blood drop on the skin surface. In doing so, a bottom surface of a finger can be lanced as described in FIG. 5. The user can then apply the blood drop from the skin surface to the test strip 1 in a number of motions, each requiring a minimal travel distance and device manipulations.

Accordingly, the embodiments of the present invention can include an analyte sensing processor 80 with an integral lancet ejector cartridge 30, and a test strip storage cartridge 75 provided on the device body 10 to store a number of test strips. As noted in FIGS. 1-4, the embodiments house the lancet ejector cartridge 30 in the recess enclosure 70 that is located at the proximal end of the device. In doing so, all of the supplies that are typically required for a test are located in the body of the device.

Most existing blood glucose meters have a separate test strip vial, and at least one existing device has the test strips mounted on a carousel for dispensing. The embodiments of the present invention described above, however, combine an analyte sensing processor 80, lancet ejector cartridge 30, and a test strip storage cartridge 75 into one device. These embodiments can include any number of variations, however, each combining a lancet ejector cartridge 30 and an analyte sensing processor 80, with provisions to store test strips in a test strip storage cartridge 75.

The embodiments of the present invention can provide any number of types of inboard, or on-device storage for a test strip storage cartridge 75 in a meter-lancet device combination, and include any number of types of retention features for the test strip storage cartridge 75, such as a mechanical locking and spring-loading ejection mechanism or other similar mechanism for engaging and retaining the test strip storage cartridge 75 in the recess enclosure 70. However, in each embodiment and versions thereof, the test strip storage cartridge 75 and recess enclosure 70 are preferably constructed so that the test strip storage cartridge 75 can be operated with ease.

The analyte sensing processor 80 measures the change in electrical current to determine the blood glucose concentration and/or other component concentrations (e.g., cholesterol, inter alia), sends a signal with the data from the measurement to the mobile device 3 which then interprets it, the reading is subsequently displayed on the graphical user interface 4 of the mobile device 3 and the display window 82 of the device body 10 to the user and also viewable and accessible on a user's online personal profile 5.

Although only a few exemplary embodiments of the apparatus and methods of the present invention have been described in detail above, those skilled in the art will readily appreciate that many modifications are possible in the exemplary embodiments without materially departing from the novel teachings and advantages of the present invention. Accordingly, all such modifications are intended to be included within the scope of the present invention as defined in the appended claims and equivalents thereof. Furthermore, these particular embodiments are merely illustrative and not restrictive.

1. An analyte sensor, comprising:
a device body having first and second ends and a surface extending between said first and second ends configured to comprise a blood glucose meter for measuring a blood glucose level of blood absorbed into a test strip; and
an analyte sensing processor for calculating and transmitting results of the measurement of the blood glucose level;
a lancet ejector cartridge disposed at said first end of said device body for storing and ejecting lancets;
a lancet port disposed at said second end of said device body;
a depth control mechanism configured for contacting the skin to be lanced and located at said first end of said device body, wherein said depth control mechanism is rotatable for setting a lancet skin penetration depth;
a test strip storage cartridge disposed at said first end of said device body for storing test strips;
a test strip exit port disposed at said second end of said device body;
a test strip entrance port, opening at said second end of said device body for positioning a test strip within said device body, adjacent to said lancet port and test strip exit port; and
a display disposed on said surface above said lancet port and said second end of said device body.

2. An analyte sensor as claimed in claim 1, wherein said device body further comprises a lancet lead-in area extending between said test strip entrance port and said lancet port.

3. An analyte sensor as claimed in claim 1, wherein said device body further comprises:
at least one operator control for data entry and review through said display.

4. An analyte sensor as claimed in claim 1. wherein said device body comprises:
a trigger button disposed on said device body for activating said lancet ejector cartridge;
an arming slide disposed on said device body for arming said lancet device; and
a test strip ejector slide disposed on said body for arming said test strip storage cartridge.

5. An analyte sensor as claimed in claim 1, wherein said body further comprises a detachable cover on said device body for providing access to said lancet ejector cartridge and said test strip storage cartridge for loading and unloading of lancets and test strips.
